(19) United States
(12) Plant Patent Application Publication
(10) Pub. No.: US 2026/0182480 P1
(43) Pub. Date: Jun. 25, 2026

(54) Strawberry Plant Named 'CBC016'

(50) ;

(71) Applicant: Kyle M VandenLangenberg, Tracy, CA (US)

(72) Inventor: Kyle M VandenLangenberg, Tracy, CA (US)

(73) Assignee: California Berry Cultivars, LLC, French Camp, CA (US)

(21) Appl. No.: 19/427,702

(22) Filed: Dec. 19, 2025

Related U.S. Application Data
(60) Provisional application No. 63/738,107, filed on Dec. 23, 2024.

(51) Int. Cl. A01H 6/74; A01H 5/08

(52) U.S. Cl. CPC A01H 6/7409; A01H 5/08
USPC PLT/208

(57) ABSTRACT

A new and distinct cultivar of strawberry plant (Fragaria x ananassa) named ‘CBC016’ is presented here. This new day-neutral strawberry cultivar is characterized by moderate vigor plants which produce high yields of large, firm, flavorful fruit throughout the growing season.

Botanical classification: Fragaria x ananassa.

Variety denomination: The new strawberry variety denomination is ‘CBC016’.

### BACKGROUND OF THE INVENTION

This invention relates to a new and distinctive day-neutral type cultivar designated as ‘CBC016’. The new cultivar was the result of a controlled cross performed in 2016 in Huelva, Spain between the cultivar ‘Fronteras’ (U.S. Plant Pat. No. 26,709) and the cultivar ‘San Andreas’ (U.S. Plant Pat. No. 19,975). ‘CBC016’ was first fruited at an experimental orchard near French Camp, California in 2017, where it was selected, originally designated ‘116.082-104’, and propagated asexually by runners. Following selection and during testing, the plant of this selection was later designated ‘CBC016’. Asexual propagules from this original source have been tested in Watsonville, California, in Oxnard, California, and to a limited extent in grower fields starting in 2021. The cultivar is stable and reproduces true to type in successive generations of asexual reproduction.

### COMPARISON WITH PARENTS

The parent ‘Fronteras’ is a short-day cultivar that was selected for its overall high appearance score, large fruit, and disease resistance. Plants of ‘Fronteras’ are similar in size compared to ‘CBC016’. The parent ‘San Andreas’ is a day-neutral cultivar that was selected for its firm conical fruit and day-neutral fruiting habit. Plants of ‘San Andreas’ are smaller than ‘CBC016’.

### SUMMARY OF THE INVENTION

‘CBC016’ is a day-neutral cultivar that produces fruit over an extended period when treated appropriately in arid, subtropical climates. When treated with appropriate planting regimes, ‘CBC016’ has larger fruit and produces individual plant yields greater than that of either parent. The following traits have been repeatedly observed over multiple testing years and locations and are determined to be unique characteristics of ‘CBC016’. These characteristics in combination distinguish ‘CBC016’ as a new and distinct Strawberry plant:
1. Medium to large sized plant.
2. Day-neutral flowering habit.
3. Medium glossy red fruit, conic shaped.
4. Fruit is firm throughout the growing season.
5. Fruit postharvest longevity is good.
6. Excellent disease resistance.

The cultivar ‘CBC016’, when planted under appropriate conditions, is a continuously flowering day-neutral type with exceptional total yields. Fruit quality is good with excellent flavor. In addition to its yield performance, ‘CBC016’ is resistant to Fusarium oxysporum f. sp fragariae, the causal agent of fusarium wilt in strawberry plants.

### BRIEF DESCRIPTION OF THE FIGURES

The accompanying color photographs, identified as FIGS. 1 through 5, show the appearance of typical specimens of the new strawberry cultivar designated ‘CBC016’. These Figures depict the colors, as nearly true as can be achieved with conventional photography.

FIG. 1 shows a typical leaf and petiole structure.

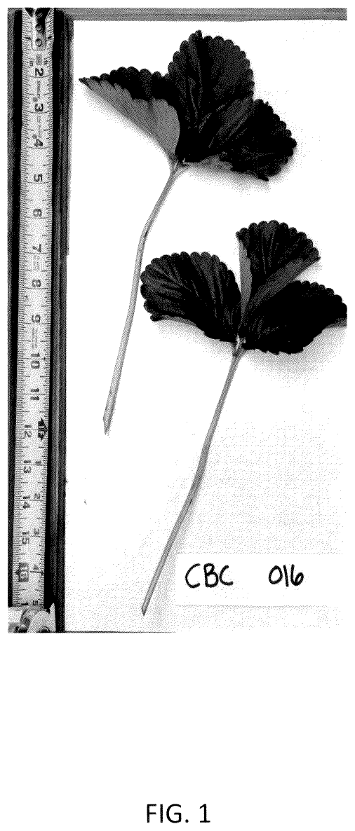

FIG. 2 shows a typical plant and fruit early season.

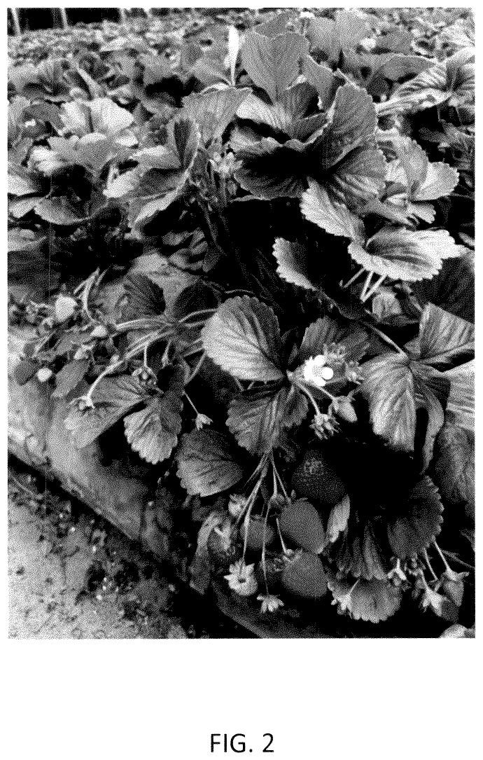

FIG. 3 shows typical fruit in a box.

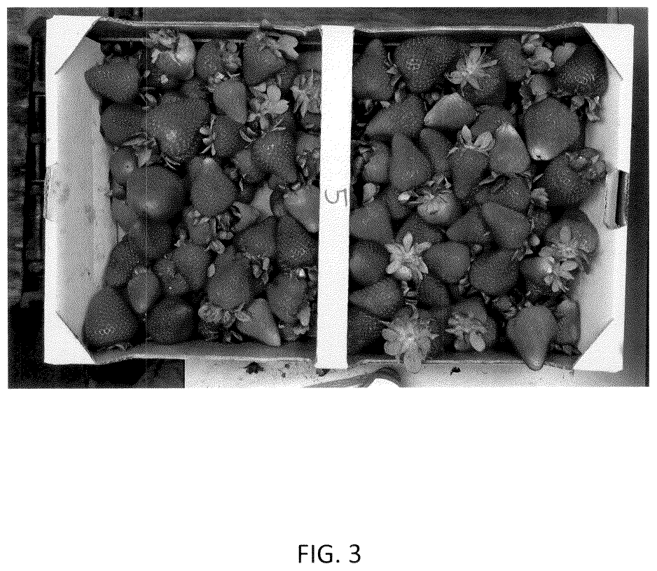

FIG. 4 shows a cross-section of representative fruit mid-season.

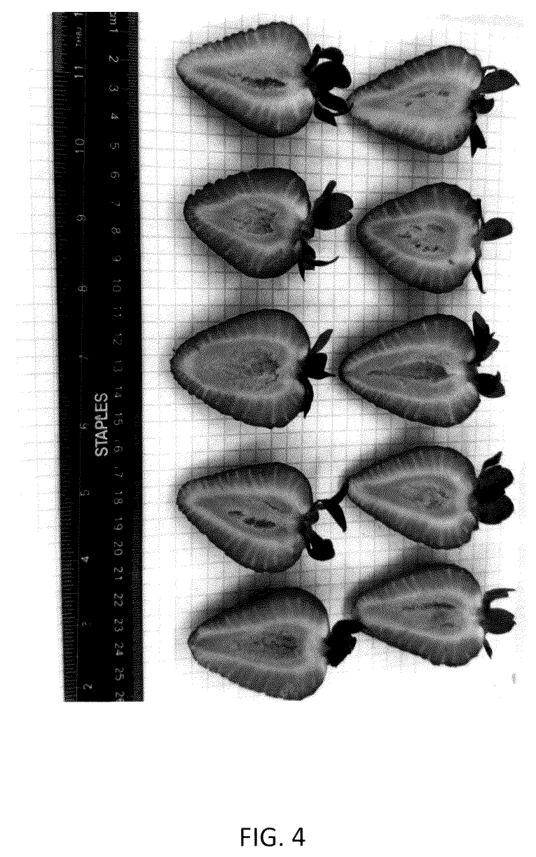

FIG. 5 shows a typical plot in the field of plants and fruit.

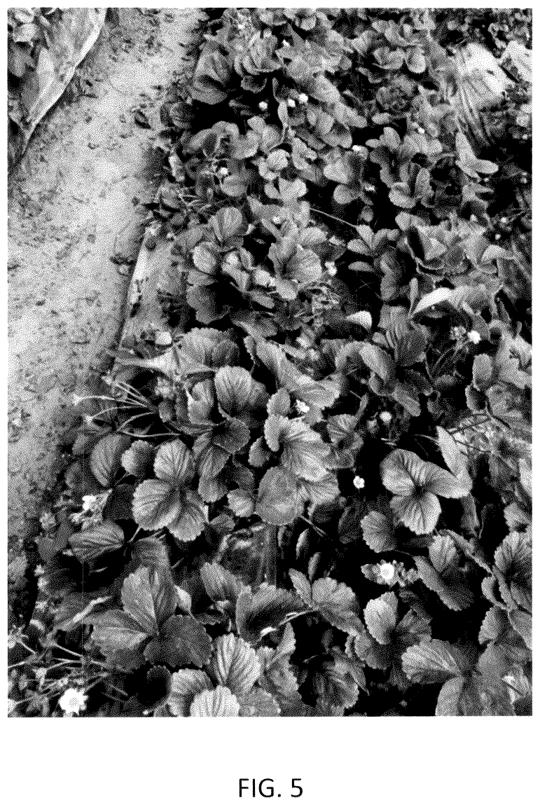

### DETAILED BOTANICAL DESCRIPTION OF THE PLANT

‘CBC016’ is a new and distinct day-neutral cultivar of strawberry, genus and species Fragaria x ananassa. Plants of ‘CBC016’ bloom continuously in higher elevation plantings as well as the heat of low elevation nursery locations such as French Camp, CA, consistent with other day-neutral varieties. The plants of the new cultivar have a moderate vigor.

‘CBC016’ exhibits several characteristics which are improvements over one or both of its parent varieties, and other cultivars known to the Inventor. These characteristics are compared with other cultivars in the following discussion and tables.

The fruit size of ‘CBC016’ is large sized, similar to or larger than its parents. The fruit size of ‘CBC016’ is still within an ideal range since it is not too large to be damaged in shipping containers, but not too small to allow for efficient picking and packing. Fruit of ‘CBC016’ is also firmer than its parents. Storability of the fruit has not been measured but in informal observations the fruit retains good physical characteristics and appearance for several days after harvest at room temperature and up to at least a week in common refrigerator conditions of roughly 3° C.

The data set for ‘CBC016’ set forth below was collected in May 2025 from plants grown at a test plot in Watsonville, CA. Yield, size, firmness data is either averaged over the entire 2024-2025 growing season or presented as monthly totals at the same location. Description data was collected on five-month-old plants.

The observed characteristics of ‘CBC016’ are set forth in Table 1, 2, 3, and 4 below. Color terminology, where noted, is in accordance with the Munsell Colorchart. The observed characteristics of ‘CBC016’ are compared to the closest cultivar known to the Inventor, in Table 2, 3, and 4. Table 5 sets forth the test results of DNA fingerprinting using eight SSR markers.

TABLE 1
Detailed Description of Characteristics of ‘CBC016’

| | |
|---|---|
| SPECIFICATION: | |
| Genus/Species | Fragaria X ananassa |
| Market Name | Strawberry |
| PARENTS: | |
| Female | ‘San Andreas’ |
| Male | ‘Fronteras’ |
| PLANT: | |
| Type | Day-neutral |
| Growth Habit | Upright |
| Foliage density | Moderate |
| Vigor | Moderate |
| Height | Average 27.7 cm; range 23-31 cm |
| Width | Average 44.7 cm; range 34-58 cm |
| Disease tolerance | Test plots have shown tolerance to |
| | Fusarium oxysporum f. sp fragariae |
| LEAF: | |
| Width | 15.5 cm |
| Color | Adaxial surface: 7.5 GY 5/7 |
| | Abaxial surface: 7.5 GY 9/4 |
| Pubescence | Slight |
| Glossiness | Medium |
| Variegation | Not present |
| TERMINAL LEAFLET | |
| Length | Average 89 mm; range 84-111 mm |
| Width | Average 70 mm; range 67-75 mm |
| Ratio (length/width) | 1.27 |
| Margins | Crenate |
| Leaflet shape | Obovate |
| Base shape | Cuneate |
| Cross-section shape | Moderately Concave |
| PETIOLE | |
| Pubescence | Slight |
| Petiole color | 5 GY 8/8 |
| Petiole length | Average 18 cm; range 15-22 cm |
| Petiole diameter | Average 4.2 mm; range 4-5 mm |
| STOLON | |
| Anthocyanin coloration | Present, slight |
| Pubescence | Slight |
| Diameter | Average 3 mm, range 2-4 mm |
| STIPULE | |
| Anthocyanin coloration | Light |
| Length | Average 11.3 mm; range 9-13 mm |
| Width | Average 6 mm; range 5-7 mm |
| INFLORESCENCE: | |
| Flowering | Normal |
| Position | At or above canopy |
| Petal arrangement | Broadly rounded; slightly cupped |
| Calyx size | Large |
| Number of blooms | 24 |
| Length | 3.8 cm |
| Flower diameter | Average 31 mm; range 28-36 mm |
| Petal number | Average 6; range 5-7 |
| Petal length | Average 13 mm; range 12-14 mm |
| Petal width | Average 11.5 mm; range 11-14 mm |
| Ratio (length/width) | 1.13 |
| Petal color | N 9.5/0 |
| Calyx color | Adaxial surface: 7.5 GY 5/7 |
| | Abaxial surface: 7.5 GY 8/7 |
| Stamens length | Average 25 mm; range 20-30 mm |
| PEDICEL | |
| Pubescence | Slight |
| Length | Average 132 mm; range 118-150 mm |
| Width | Average 3 mm; range 2-4 mm |
| Ratio (length/width) | 44 |
| Color | 5 GY 8/8 |
| FRUIT: | |
| Bearing | Day-neutral |
| Individual fruit weight | Seasonal average: 31.8 g |
| Shape | Long conic |
| Achenes | Sunken |
| Glossiness | High gloss |
| External color evenness | Moderately even |
| External color | 7.5 R 4/11 |
| Internal color | 10 R 8/6 |
| Band at top without achenes | Narrow |
| Fruit Yield | Grams per plant: 2172 g |
| Firmness | 0.61 kg/force |
| Achene color | 10 Y 9/9 |

TABLE 2
Marketable yield of ‘CBC016’ compared to other cultivars known to the
Inventor in Watsonville, CA during the 2024-2025 season:
‘Cabrillo’, U.S. Plant Pat. No. 27,830 and
‘Monterey’, U.S. Plant Pat. No. 19,767.

| | Marketable yield (g/plant) | | | | | |
|---|---|---|---|---|---|---|
| Cultivar | Apr. | May | Jun. | Jul. | Aug. | Total |
| ‘CBC016’ | 237 | 524 | 652 | 608 | 151 | 2172 |
| ‘Cabrillo’ | 186 | 415 | 717 | 800 | 218 | 2336 |
| ‘Monterey’ | 195 | 485 | 648 | 600 | 178 | 2105 |

TABLE 3
Size of ‘CBC016’ compared to other cultivars known
to the Inventor in Watsonville, CA during the 2024-2025 season.

| | Fruit size (g/fruit) | | | | | |
|---|---|---|---|---|---|---|
| Cultivar | Apr. | May | Jun. | Jul. | Aug. | Average |
| ‘CBC016’ | 43.9 | 38.2 | 29.6 | 25.2 | 22.0 | 31.8 |
| ‘Cabrillo’ | 37.0 | 37.9 | 33.5 | 27.9 | 23.6 | 32.0 |
| ‘Monterey’ | 41.2 | 39.2 | 30.0 | 23.2 | 19.7 | 30.7 |

TABLE 4
Firmness of ‘CBC016’ compared to other cultivars known
to the Inventor in Watsonville, CA during the 2024-2025 season.
Firmness (kg/force)

| Cultivar | May | Jun. | Jul. | Average |
|---|---|---|---|---|
| ‘CBC016’ | 0.68 | 0.58 | 0.57 | 0.61 |
| ‘Cabrillo’ | 0.60 | 0.55 | 0.56 | 0.57 |
| ‘Monterey’ | 0.56 | 0.51 | 0.50 | 0.52 |

TABLE 5
DNA finger printing results of eight SSR markers.

Allelic Fingerprint Analysis

| Cultivar | M1 | M2 | M3 | M4 | M5 | M6 | M7 | M8 |
|---|---|---|---|---|---|---|---|---|
| ‘CBC016’ | 202, | 259, | 188, | 279, | 154, | 242, | 121, | 213, |
| | 212, | 264, | 190, | 281, | 156 | 251, | 124, | 215, |
| | 222 | 271, | 216, | 289, | | 257, | 130, | 228 |
| | | 275 | 232 | 292 | | 262 | 146 | |

1. A new and distinct strawberry plant named ‘CBC016’ as described and illustrated herein.
